(19) United States
(12) Patent Application Publication
(10) Pub. No.: US 2016/0331320 A1
(43) Pub. Date: Nov. 17, 2016

(54) ADAPTIVE WEARABLE SMART FABRIC

(71) Applicant: HCL Technologies Limited, Noida (IN)

(72) Inventors: Gopi Krishna Durbhaka, Hyderabad (IN); Satya Sai Prakash Kanakadandi, Hyderabad (IN); S U M Prasad Dhanyamraju, Hyderabad (IN)

(21) Appl. No.: 15/080,360

(22) Filed: Mar. 24, 2016

(30) Foreign Application Priority Data
May 15, 2015 (IN) 1364/DEL/2015

(51) Int. Cl. A61B 5/00; A61B 5/0205; G05B 19/406; A41D 1/00

(52) U.S. Cl. CPC A61B 5/6804; A41D 1/002; A61B 5/7264; A61B 5/0205; G05B 19/406; A61B 2562/0219; A61B 5/02438

(57) ABSTRACT

The present disclosure discloses an adaptive wearable smart fabric. The adaptive wearable smart fabric may comprise sensors being accelerometer sensor, load sensor and pulse sensor. The accelerometer sensor and load sensor are adapted to sense the posture data and movement data of the user of an adaptive wearable smart fabric. The sensors are coupled with microcontroller that captures the sensed data and determines the posture based on analytic model. The microcontroller may be further coupled with PID controller and air pump which may inflate and deflate the air diaphragm placed within the fabric. The inflation and deflation of air diaphragm is dynamically controlled to provide comfort to the user of an adaptive wearable smart fabric.

### CROSS-REFERENCE TO RELATED APPLICATIONS AND PRIORITY

The present application claims benefit from Indian Complete Patent Application No. 1364/DEL/2015, filed on May 15, 2015, the entirety of which is hereby incorporated herein by reference for all purposes.

### TECHNICAL FIELD

The present subject matter described herein, in general, relates to an inflatable-deflatable adaptive wearable smart fabric.

### BACKGROUND

For long, fabrics have formed part of fashion and clothing only. Standard fabrics have property according to their type and construction and are maintained despite any change in ambient condition and/or physical activity. However, with evolving technology and growing popularity of wearable computing devices, fabrics no more have the standard utility but are employed with information technology to construct smart fabric. Smart fabrics include incorporation of digital devices which are incorporated or attached to the fabric to produce a particular effect based on external factors and environment. Examples of some of smart fabrics available today are: activity regulated clothes which change temperature in response to extreme conditions, sanitized fabrics for sportswear that contain anti-bacterial properties to combat smell and sweat, fibre optics woven into garments to act as radios or mp3 players and lights incorporated into clothing for safety purposes.

The conventional use of smart fabrics have been in the medical or sports industry. Usually smart fabrics in medical or sports industry are used to monitor vital body signs of the wearer including heart rate, respiration rate, body temperature and blood pressure etc. It may be understood that with the growing need of smart wearable fabrics there exists a wide applicability to aid the wearer comfort and security.

### SUMMARY

This summary is provided to introduce aspects related to adaptive wearable smart fabric which is further described below in the detailed description. This summary is not intended to identify essential features of subject matter nor is it intended for use in determining or limiting the scope of the subject matter.

In one implementation, an adaptive wearable smart fabric is disclosed. The adaptive wearable smart fabric may comprise one or more sensors adapted to sense posture data and movement data of a user wearing the adaptive smart fabric. The adaptive wearable smart fabric may further comprise a processor coupled with a memory storing instructions. The processor may execute the instructions stored in the memory. In one embodiment, the processor may execute an instruction in order to capture the posture data and the movement data from the one or more sensors. Further, the processor may execute an instruction in order to determine, using an analytics model, posture of the user wearing an adaptive smart fabric based upon the posture data and the movement data captured from the one or more sensors. The adaptive smart fabric may further comprise a Proportional-Integral-Derivative (PID) flow controller coupled with the processor. The PID flow controller may be configured to dynamically control, via a combination of air pump and a valve, inflation or deflation of an air diaphragm placed within the fabric. The air diaphragm may be inflated or deflated based on the posture of the user wearing an adaptive smart fabric.

In another implementation, a method executed in an adaptive wearable smart fabric is disclosed. In one aspect, the method may comprise capturing, by a processor, posture data and the movement data from one or more sensors. Further, the method may comprise determining, by a processor, using an analytic model, posture of the user wearing an adaptive smart fabric based upon the posture data and the movement data captured from the one or more sensors. Further, the method may comprise controlling, via a Proportional-Integral-Derivative (PID) controller coupled with the processor, air for dynamic inflation or deflation of an air diaphragm placed within the fabric. In one aspect, the air diaphragm is inflated or deflated based on the posture of the user wearing an adaptive smart fabric.

### BRIEF DESCRIPTION OF THE DRAWINGS

The foregoing detailed description of embodiments is better understood when read in conjunction with the appended drawings. For the purpose of illustrating the disclosure, there is shown in the present document example constructions of the disclosure. However, the disclosure is not limited to the specific methods and apparatus disclosed in the document and the drawings.

The detailed description is described with reference to the accompanying figures. In the figures, the left-most digit(s) of a reference number identifies the figure in which the reference number first appears. The same numbers are used throughout the drawings to refer like features and components.

FIG. 1 illustrates a detailed architectural layout of the adaptive wearable smart fabric, in accordance with an embodiment of the present disclosure.

FIG. 2 illustrates a conventional Adaptive Resonance Theory (ART) based model flowchart.

FIG. 3 illustrates a learning Adaptive Resonance Theory (ART) model flowchart.

FIG. 4 illustrates an example posture of the user of the adaptive wearable smart fabric, in accordance with an embodiment of the present disclosure.

FIG. 5 illustrates a 3D plane to compute the aerodynamic equilibrium, in accordance with an embodiment of the present disclosure.

FIG. 6 illustrates a generalized setup of the sensor within the fabric, in accordance with an embodiment of the present disclosure.

FIG. 7 illustrates a method flowchart to facilitate inflation and deflation of adaptive wearable smart fabric with aerodynamic control, in accordance with an embodiment of the present disclosure.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that the singular forms “a,” “an,” and “the” include plural references unless the context clearly dictates otherwise. Although any systems and methods similar or equivalent to those described herein can be used in the practice or testing of embodiments of the present disclosure, the exemplary, systems and methods are now described. The disclosed embodiments are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated, but is to be accorded the widest scope consistent with the principles and features described herein. It is understood that the system and configuration of the adaptive wearable smart fabric are described in the context of the following exemplary system.

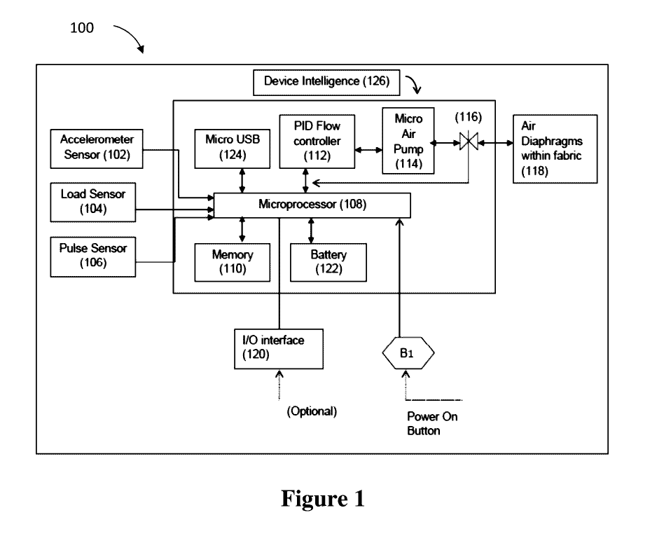

Referring to FIG. 1, detailed architectural layout of the adaptive wearable smart fabric 100 is shown, in accordance with an embodiment of the present disclosure. Further, the adaptive wearable smart fabric 100 is of such shape and type that may be worn on various parts of the body of a wearer. In one embodiment, the adaptive wearable smart fabric 100 comprises of sensors embedded at one or more locations within the fabric. The sensors may comprise an Accelerometer Sensor 102, a Load Sensor 104 and a Pulse Sensor 106. The adaptive wearable smart fabric 100 may further comprise a processor 108, a memory 110, a Proportional-Integral-Derivative (PID) flow controller 112, a micro air pump 114, an air flow control 116, an air diaphragm 118 and an input/output (I/O) interface 120. It must be understood that FIG. 1 illustrates only a single air diaphragm, however the scope of the present disclosure is extended to several such air diaphragms placed at different positions within the adaptive wearable smart fabric 100. The architectural layout may further comprise a Battery 122 and Micro USB 124.

The processor 108 may be implemented as one or more microprocessors, microcomputers, microcontrollers, digital signal processors, central processing units, state machines, logic circuitries, and/or any devices that manipulate signals based on operational instructions. Among other capabilities, the at least one processor 108 is configured to fetch and execute computer-readable instructions stored in the memory 110.

The I/O interface 120 may include a variety of software and hardware interfaces, for example, a web interface, a graphical user interface, and the like. The I/O interface 120 may allow the processor 108 to interact with the user of the adaptive wearable smart fabric.

The memory 108 may include any computer-readable medium and computer program product known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

According to embodiments of present disclosure, the processor 108 may be communicatively coupled with the sensors placed at one or more positions within the fabric. In one aspect, the accelerometer sensor 102 coupled with the processor 108 may be capable of determining the velocity or motion of the user of the adaptive wearable smart fabric. The velocity or motion may be determined by the multiple such accelerometer sensor(s) 102 placed within the fabric. The accelerometer sensor(s) 102 are placed in such a manner that facilitates the accelerometer sensor(s) 102 to measure the change in posture and orientation of the user of the adaptive wearable smart fabric 100. The accelerometer sensor(s) 102 may be in communication, either wired or wirelessly. In one aspect of the invention, the accelerometer sensor 102 may be a 3-axis (triple-axis) accelerometer.

In one aspect, the processor 108 may be further communicatively coupled with the load sensor 104. The load sensor 104 may be configured to determine stress or force points of the user of the adaptive wearable smart fabric 100. The force points may be determined by multiple such load sensor(s) 104 placed within the fabric. Similar to accelerometer sensor 102, the load sensor(s) 104 are placed within the fabric in a manner that facilitates the load sensor(s) 104 to measure the stress parts of the user of the adaptive wearable smart fabric 100. The load sensor(s) 104 may be in communication, either wired or wirelessly. The standard load sensors 104 are of a nature that may convert the stress measurable by a magnitude of the electrical signals generated.

In one aspect, the processor 108 may be further communicatively coupled with the pulse sensor 106 to determine the vital signs of the user of the adaptive wearable smart fabric. The vital signs may be determined by the multiple such pulse sensor(s) 106 placed at specified locations within the fabric. Similar to accelerometer sensors 102, the pulse sensors 106 are placed within the fabric in a manner that facilitates the pulse sensors 106 to monitor the vital signs of the wearer. The pulse sensors 106 may be in communication, either wired or wirelessly. In one aspect of the invention, the vital signs sensed by the pulse sensors 106 may include heart rate, blood pressure, body temperature etc. Further, the pulse sensors 106 may be of a nature to identify deviation from the normal bodily conditions and may alert the user of the adaptive wearable smart fabric or the caregiver of deviation of normal bodily conditions.

In one aspect of the invention, the accelerometer sensor 102 and the load sensor 104 may transmit the captured data to the processor 108 for further analysis. Based upon the data received from the accelerometer sensor 102 and the load sensor 104, the processor 108 may analyze the data so as to determine the posture of the user of the adaptive wearable smart fabric 100. The processor 108 may classify the posture to be one of sitting, standing or sleeping. The classification may be further categorized as manner of sitting, standing or sleeping. In an example, the user of the adaptive wearable smart fabric 100 may either be sleeping as back down, sleeping as stomach down or sleeping on side. To determine and classify the posture of user of the adaptive wearable smart fabric, an analytic model such as neural networks may be utilized. The neural networks may preferably be based on an Adaptive Resonance Theory (ART).

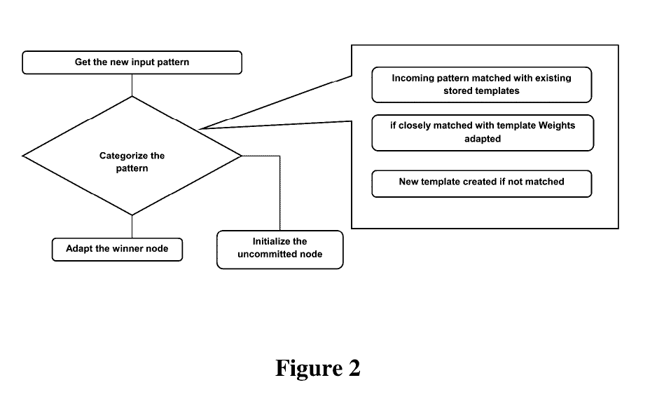

It is known in the art that ART utilizes unsupervised learning and are available from several sources for pattern recognition and prediction. ART is capable of self-organizing its modes in real time producing the stable recognition while retaining its ability to learn new patterns apart from preserving the regular trained knowledge. ART, in the present invention, overcomes Stability-Plasticity Dilemma (SPD) while learning a new case and also remains stable in spite of being adaptive (plasticity) to the new occurring inputs. The FIG. 2 illustrates a conventional Adaptive Resonance Theory (ART) based model flowchart.

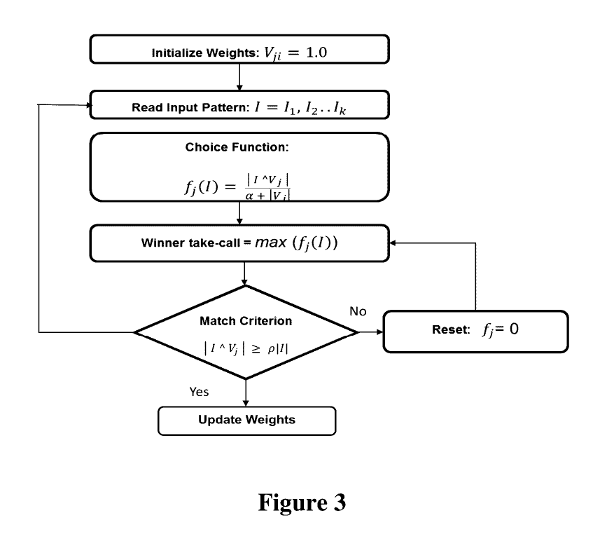

In general, ART neural networks are implemented using analytical solutions or approximations to the differential equations. The unsupervised ART neural networks are basically similar to many iterative clustering algorithms in which each case is processed by finding the ‘nearest’ cluster to the input. ART neural networks are defined algorithmically typically consisting of a comparison field and recognition field composed of neurons, a vigilance parameter (ρ), and a reset function. The comparison field receives the input and transfers it to its best match in recognition field. The input is best matched when the single neuron whose set of weights matches closely. The recognition field allows each neuron to represent a category to which inputs are classified. Once classified, the reset compares the strength of the recognition match to the vigilance parameter (ρ). To compute the strength of the recognition match the choice function measures the degree of resemblance of input and weights. The vigilance parameter (ρ) uses winner take-all learning strategy. Further, the match criteria measure the resonance likeness of input and weights. The function is used in conjunction with the vigilance parameter (ρ). Where for a good resonance the match criteria should be greater than the vigilance parameter (ρ). On meeting the vigilance threshold the training commences. In the search procedure neurons are disabled one by one by the reset function until the vigilance parameter (ρ) is satisfied by a recognition match. If no existing neuron is matched to an uncommitted neuron is committed and adjusted towards matching the input, the phenomenon is termed as plasticity. It is to be noted that no existing neuron are deleted by the introduction of new inputs or new neurons. FIG. 3 illustrates a learning Adaptive Resonance Theory (ART) model flowchart. With the context of the present invention, the different cluster/input may be interpreted as representing different postures of the user of the adaptive wearable smart fabric.

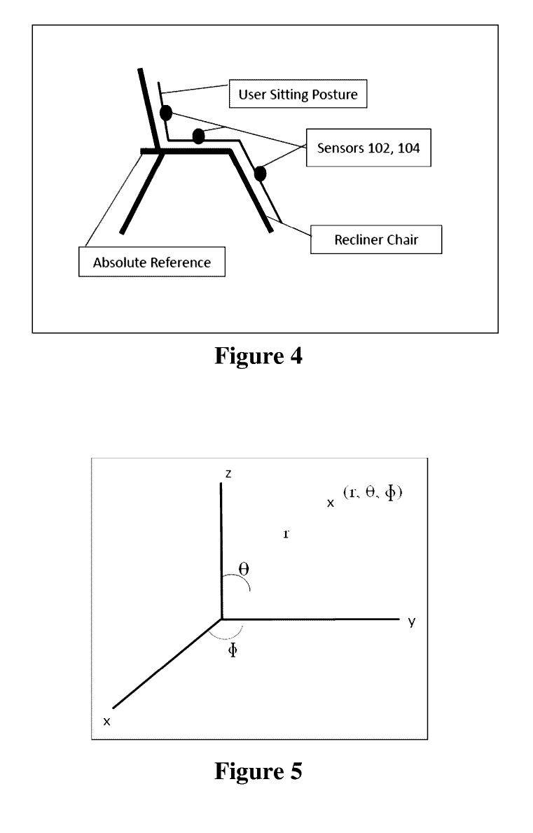

With the present disclosure, no extensive computing is required using ART to determine the posture. The neural network may be trained to recognize every known posture in general of the intended user by the manufacturer as a template. Further, the neural network may receive the input from the sensor device such as the accelerometer sensor 102 and the load sensor 104 or any other sensor that may provide the posture data and movement data of a user as input data. To classify the input data, the input data is compared with the existing template. If the input data closely matches the posture template then the posture is so determined. In a condition that the input data is not a match to the input data then the neural network may organize itself in an unsupervised manner to identify new inputs and train the new input data. In an example situation, the neural network may be trained to recognize one type of sitting posture termed as a template/neuron from a variety of sitting postures with respect to the sitting tool such a chair. It is to be noted that there may be several sitting postures based on the type of sitting tool or the manner of sitting. FIG. 4 illustrates a user of the adaptive wearable smart fabric in a sitting posture on a recliner chair wherein the sensors 102, 104 provide the input data. The absolute reference in accordance with the FIG. 4 is the recliner chair against which the posture of the user is determined. On receiving the inputs from the accelerometer sensor 102 and the load sensor 104 or any other sensor that may provide the posture data and movement data of a user as input data, the input data is best matched in the recognition field. Further, the choice function measures the degree of resemblance of input and weights of the neuron to determine the strength of the recognition. The match criteria further measures the resonance likeness of input and weights. If the match criteria is greater than the vigilance parameter (ρ), then the training commences i.e. the user of the adaptive wearable device is in a sitting posture which exists as a trained template. Contrary, if the input data doesn't match the template/neuron, a new uncommitted neuron is committed and adjusted towards matching the input data. This new neuron forms a part of the template for recognition for further received input data, hence adaptive in nature. In this manner the posture of the user of the adaptive wearable fabric is determined using ART.

In accordance with an embodiment, once the posture is determined using ART, the processor 108 further quantifies the air required by the air diaphragms 118 placed within the smart fabric. The air diaphragm(s) 118 may be placed at specific distance within the fabric. The processor 108 is coupled with the Proportional-Integral-Derivative (PID) flow controller 112 which may maintain the set point for air based on the posture. The PID flow controller 112 may be a standard PID controller or a programmable logic controller (PLC) or a panel-mounted digital controller. Further, the set point of the air is dependent upon the posture and the aerodynamic equilibrium of the user of the adaptive wearable smart fabric 100.

In accordance with an embodiment, the micro/mini dc air pump 114 along with the solenoid valve 116 connected with the PID flow controller 112 may be used to regulate the air flow in the air diaphragms 118 based on the posture of the user determined by ART. In the present disclosure, the air pump 114 and the PID flow controller 112 may be connected to the power source such as a battery. The air pump 114 further may dynamically inflate and deflate the one or more air diaphragms 118 placed at one or more locations within the fabric based on the posture and movement of the user after wearing the smart wearable fabric.

Further, the air diaphragms 118 may vary in size and shape. In a preferred embodiment the air diaphragms 118 may be cylindrical in shape. It is to be noted that the pressure in the air diaphragms 118 is maintained at constant in a manner so as to vary with respect to the movement of the user of the adaptive wearable smart fabric. The inflation and deflation of the air diaphragms 118 is in a manner so as to provide comfort to the user of the adaptive wearable smart fabric. The volume (V) of air diaphragms 118 shall vary with respect to the movement. The volume (V) of air diaphragm 118 may be calculated in a below manner:

Volume (V)=πr² Equation (1)

In one aspect of the present disclosure, the Equation 2 may be employed to determine the aerodynamic equilibrium. The aerodynamic equilibrium with respect to the referential axis depending on the various types of commutes and postures to derive the amount of air flow for inflation and deflation may be calculated by Equation 2.

Σᵢ₌₁ⁿ(kᵢ×πr²l)=½(b×h) Equation (2)

Wherein, kᵢ is the index of the cylindrical air diaphragms, r is the Radius of the air diaphragms which shall be constant i.e. varying within a specific range only, 1 is the height/length of the cylindrical air diaphragms which shall be constant for a specific range, b is the base and h is the height of the triangular vacuum. Further, it is to note that a triangular vacuum position is formed due to inclination made by the user of the adaptive wearable smart fabric due to the relative movement of the user in comparison with the absolute reference. Particular to the invention, the absolute reference is the tool against which the posture of the user is determined. In one aspect the tool for absolute reference may be a chair if the user of the adaptive wearable smart fabric is in sitting posture or a bed if the user of the adaptive wearable smart fabric is in sleeping posture. The polar coordinates illustrated in FIG. 4 represent the referential axis in the 3D which is unique to each commute. As in the FIG. 4, the [x,y,z] are the reference axis and the projected line with (r, θ, φ) is the inclination made by the user of the adaptive wearable smart fabric. In one aspect, the polar coordinates may be represented as below:

x=r sin θ* cos φ), y=r sin θ* sin φ), z=r cos θ,

r=(x²+y₂+z²)^{1/2}, θ=tan⁻¹(z/(x²+y²)^{1/2}), φ=tan⁻¹(y/x).

Further, in accordance with the embodiment, the equilibrium to start and stop the air flow is controlled by solenoid valve 116 which for different postures is based on the maximum air the diaphragm may withstand at the particular position and posture level. In one aspect of the invention, the inflation and deflation of the air diaphragms 118 may be controlled by the user of the adaptive wearable smart fabric. The periodic usage of the fabric aids the analytic model to get trained and enable the user to regulate the air flow to inflate and deflate accordingly.

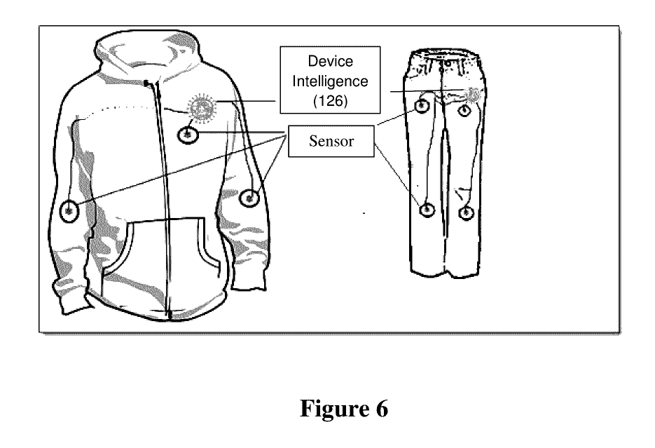

Referring to FIG. 6, a generalized setup of the sensor within the fabric in accordance with an embodiment of the present disclosure is illustrated. The sensors are placed at different locations in the fabric however not necessarily at the positions illustrated in the figure. As explained in the architectural layout of the sensors are coupled to the device intelligence 124.

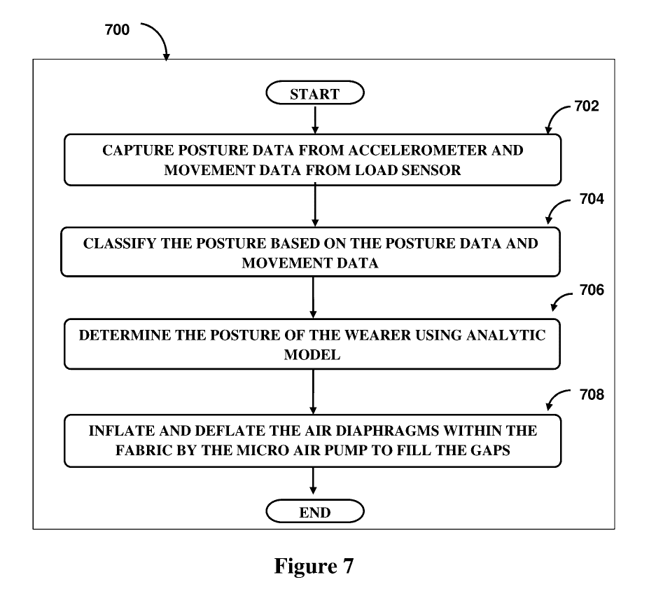

Referring now to FIG. 7, the method of inflating and deflating air diaphragms 118 of an adaptive wearable smart fabric based on the posture of the user of an adaptive wearable smart fabric is shown, in accordance with an embodiment of the present disclosure. The order in which the method 700 is described is not intended to be construed as a limitation, and any number of the described method blocks can be combined in any order to implement the method 700 or alternate methods. Additionally, individual blocks may be deleted from the method 700 without departing from the spirit and scope of the subject matter described herein. Furthermore, the method can be implemented in any suitable hardware, software, firmware, or combination thereof. However, for ease of explanation, in the embodiments described below, the method 700 may be considered to be implemented in the above described architecture layout.

At block 702, the accelerometer sensor 102 and load sensor 104 placed at one or more positions within the fabric may capture the posture data and the movement data respectively of the user of an adaptive wearable smart fabric.

At block 704, the posture of the user of an adaptive wearable smart fabric may be classified based on the posture data and the movement data.

At block 706, the posture of the user of an adaptive wearable smart fabric may be determined. The postures may be determined by using analytic model to further inflate and deflate the fabric accordingly.

At block 708, the air diaphragms 118 placed at one or more positions within the fabric may be inflated and deflated based on the determined posture by the micro air pump 114 to fill the gaps.

Although implementations of an adaptive wearable smart fabric and method have been described in language specific to structural features and/or methods, it is to be understood that the appended claims are not necessarily limited to the specific features or methods described. Rather, the specific features and methods are disclosed as examples of implementations for constructing a wearable adaptable smart fabric.

Exemplary embodiments discussed above may provide certain advantages. Though not required to practice aspects of the disclosure, these advantages may include those provided by the following features.

Some embodiments enable adaptive wearable smart fabric to be disjoint and not necessarily a full body wearable fabric.

Some embodiments enable adaptive wearable smart fabric to dynamically inflate and deflate based on different postures without any human intervention.

Some embodiments enable adaptive wearable smart fabric to provide proper sitting and sleeping ergonomics thus reducing strain on muscles and provide comfort to the user of the adaptive wearable smart fabric.

Some embodiments enable adaptive wearable smart fabric to be light weight and flexible to fold and carry.

Some embodiments enable adaptive wearable smart fabric to monitor vital signs, wherein the vital signs may be heart rate of the user of the adaptive wearable smart fabric and alert the user of the adaptive wearable smart fabric or nurse or doctor for any abnormalities.

We claim:

1. An adaptive wearable smart fabric comprising:
one or more sensors adapted to sense posture data and movement data of a user wearing an adaptive smart fabric;
a processor coupled with a memory configured to store instructions, wherein the processor executes the instructions in order to:
capture the posture data and the movement data from the one or more sensors; and
determine, using an analytic model, posture of the user wearing an adaptive smart fabric based upon the posture data and the movement data captured from the one or more sensors; and
a Proportional-Integral-Derivative (PID) controller coupled with the processor, wherein the PID controller is configured to dynamically control, via a combination of air pump and a valve, inflation or deflation of an air diaphragm placed within the fabric, and wherein the air diaphragm is inflated or deflated based on the posture of the user wearing an adaptive smart fabric.

2. The adaptive wearable smart fabric of claim 1, wherein the analytic model is based on Adaptive Resonance Theory (ART).

3. The adaptive wearable smart fabric of claim 1, wherein the one or more sensors comprises an accelerometer sensor to capture the posture data.

4. The adaptive wearable smart fabric of claim 1, wherein the one or more sensors comprises a load sensor to capture the movement data.

5. The adaptive wearable smart fabric of claim 1, wherein the one or more sensors further comprises a pulse sensor to capture the vital signs of the user wearing an adaptive smart fabric wherein the vital signs are pulse heart rate.

6. The adaptive wearable smart fabric of claim 1, wherein the inflation and deflation of the air diaphragms is such that the inflation and deflation maintains aerodynamic equilibrium.

7. The adaptive wearable smart fabric of claim 1, wherein the posture data captured comprises of standing, sitting and sleeping.

8. The adaptive wearable smart fabric of claim 1, wherein the one or more air diaphragms are placed within the fabric surrounding the neck collar, neck shoulders, underneath knees, lower back near spine, beneath waist, around waist, underneath legs, around ankle and underneath head.

9. A method comprising:
sensing, via one or more sensors, posture data and movement data of a user wearing an adaptive smart fabric;
capturing, by a processor, the posture data and the movement data from one or more sensors;
determining, by the processor, using an analytic model, posture of the user wearing an adaptive smart fabric based upon the posture data and the movement data captured from the one or more sensors; and
controlling, by a Proportional-Integral-Derivative (PID) controller coupled with the processor, air for dynamic inflation or deflation of an air diaphragm placed within the fabric, and wherein the air diaphragm is inflated or deflated based on the posture of the user wearing an adaptive smart fabric.
